(19) United States
(12) Patent Application Publication
(10) Pub. No.: US 2014/0315727 A1
(43) Pub. Date: Oct. 23, 2014

(54) METHOD FOR MULTIPLEXED MOLECULAR DETECTION

(71) Applicant: Base Pair Biotechnologies, Inc., Houston, TX (US)

(72) Inventors: George W. Jackson, Pearland, TX (US); Mark Morris, Houston, TX (US)

(73) Assignee: Base Pair Biotechnologies, Inc., Houston, TX (US)

(21) Appl. No.: 14/257,943

(22) Filed: Apr. 21, 2014

Related U.S. Application Data
(60) Provisional application No. 61/813,642, filed on Apr. 19, 2013.

(51) Int. Cl. C12N 15/10

(52) U.S. Cl. CPC C12N 15/1072
USPC 506/2; 506/9

(57) ABSTRACT

Molecular probes to particular targets may be nucleic acids that may generally possess resistance to degradation when bound to a target molecule. For example, the molecular probes may be generally resistant to nuclease degradation when bound to their target molecules, and generally not resistant to nuclease degradation when unbound to their target molecules. This may be utilized, for example, to selectively degrade unbound molecular probes while preserving the bound molecular probes, which may thus serve as an indication of the presence of their target molecules in a sample.

### CROSS-REFERENCE TO RELATED APPLICATIONS

This application claims the priority and benefit of U.S. provisional patent application Ser. No. 61/813,642, filed Apr. 19, 2013, entitled “METHOD FOR MULTIPLEXED MOLECULAR DETECTION”, the contents of which is hereby incorporated by reference in its entirety.

### FIELD OF THE INVENTION

This invention relates to methods and compositions for molecular detection, for example, to methods and compositions utilizing target-specific molecular probes.

### BACKGROUND OF THE INVENTION

Proteomics is often considered the next step in the study of biological systems, after genomics. The challenge of unraveling the proteome is generally considered much more complicated than genomics, primarily because the proteome differs from cell to cell and constantly changes through biochemical interactions with the genome and the environment. An organism has radically different protein expression in different parts of its body, different stages of its life cycle and different environmental conditions. Another major difficulty is the complexity of proteins relative to nucleic acids; in humans there are about 25,000 identified genes but an estimated ˜500,000 proteins derived from these genes. This increased complexity derives from mechanisms such as alternative splicing, protein modification (glycosylation, phosphorylation) and protein degradation. The level of transcription of a gene gives only a rough estimate of its level of expression into a protein. An mRNA produced in abundance may be degraded rapidly or translated inefficiently, resulting in a small amount of protein. Additionally, many proteins experience post-translational modifications that profoundly affect their activities; for example some proteins are not active until they become phosphorylated. Methods such as phosphoproteomics and glycoproteomics are used to study post-translational modifications. Many transcripts also give rise to more than one protein through alternative splicing or alternative post-translational modifications. Finally, many proteins form complexes with other proteins or RNA molecules, and only function in the presence of these other molecule.

Over the years, antibody-mediated detection has proven to be one of the most robust and sensitive assays for any non-nucleic-acid target. Small-molecule toxins and other bioactive compounds, important protein “biomarkers” indicating disease and/or pathogen activity, and even whole viral capsids can be readily detected and quantified by immunoassays. Despite incredible successes, antibody-based diagnostics suffer several well-recognized drawbacks.

### SUMMARY OF THE INVENTION

This invention relates to methods for molecular detection, for example, to methods for molecular detection utilizing target-specific molecular probes. In general, the target-specific molecular probes may be used to detect the presence or absence of a specific target or targets, such as in a mixture or sample. The target-specific molecular probes may bind to a particular target with relatively high affinity.

In general, a target molecule may refer to any appropriate targets, which may include, but are not limited to atomic/ionic targets, molecular targets, biomolecules, proteins, molecular complexes, cells, tissues, viruses, and/or any other appropriate target or combinations thereof.

In exemplary embodiments, target-specific molecular probes include substantially single-stranded nucleic acids and/or modifications thereof. In general, a molecular probe may bind with relatively high specificity to a given target and an example may be or include an aptamer. Aptamers may generally include, but are not limited to, single-stranded nucleic acid, such as, for example, single-stranded DNA (ssDNA), single-stranded RNA (ssRNA), and/or a combination thereof; at least a portion of double-stranded nucleic acid, such as, for example, double-stranded DNA (dsDNA), double-stranded RNA (dsDNA), and/or combinations thereof; modified nucleotides and/or other useful molecules, moieties, and/or other functional chemical components, or combinations thereof; or combinations thereof or similar.

In one embodiment of the invention, a plurality of molecular probes for a plurality of different target molecules may be mixed with a sample. When target molecules to any of the molecular probes are present in the sample, the molecular probes may generally bind with affinity to the target molecules present.

In an exemplary embodiment, the molecular probes may be nucleic acids that may generally possess resistance to degradation when bound to a target molecule. For example, the molecular probes may be generally resistant to nuclease degradation when bound to their target molecules, and generally not resistant to nuclease degradation when unbound to their target molecules. This may be utilized, for example, to selectively degrade unbound molecular probes while preserving the bound molecular probes, which may thus serve as an indication of the presence of their target molecules in a sample.

Since nucleases are present in many environments and potential samples, it may be generally desirable to prevent premature degradation of the molecular probes prior to contacting them with a sample, and also to prevent premature degradation due to nucleases which may be present in the samples themselves. The use of aptamers in vivo or in cell culture is generally challenged by the susceptibility of unmodified nucleic acids to degradation by nucleases. In particular 3′-exonuclease activity has been found to be the most prevalent nuclease activity both in calf and human serum. The degradation of unmodified DNA oligonucleotides in serum generally begins within an hour after administration, and that the oligonucleotide may be completely removed within 24h.

In some embodiments, molecular probes may be synthesized with a 3′-inverted thymidine, and/or other modified 3′-bases which may generally be incompatible with 3′-nuclease activity, and thus may resist degradation. Inhibition of nuclease activity may also be achieved by addition of EDTA and/or other chelating agents to the sample, which may sequester metal ions necessary for nuclease activity.

In an exemplary aspect, the unbound molecular probes may be degraded and/or digested using a nuclease which may degrade free, unbound nucleic acids. In embodiments where the molecular probes possess 3′-modifications to resist nuclease activity, a 5′-acting nuclease may be utilized, such as, for example, E. coli exonuclease VII, which digests DNA in both the 5′→3′ and the 3′→5′ directions. In general, the selected nuclease may generally not degrade and/or otherwise significantly affect molecular probes bound to their target molecules.

In some embodiments, the molecular probes bound to their target molecules may be linked to their target molecules with durable linkages such that the molecular probes may better resist nuclease degradation when bound to the target molecules. In one embodiment, a nucleic acid molecular probe bound to a target molecule may be reversibly cross-linked using, for example, a reversible formaldehyde crosslink. This may generally improve resistance of the nucleic acid molecular probe to a nuclease. The reaction mixture may then be treated with a nuclease to digest any unbound molecular probes such that they do not interfere with the detection assay by producing false positives and/or any other undesirable reaction. The crosslink may then be later removed such that the molecular probe may be dissociated from the target molecule for amplification.

In an exemplary aspect, the bound, undigested molecular probes may generally indicate the presence of their particular target molecules and may also indicate the relative stoichiometric amount based on the amount of undigested molecular probes. This may be utilized to detect and/or quantify the present target molecules in a sample, such as, for example, by identifying, amplifying and/or quantifying the undigested molecular probes.

In an exemplary embodiment, a quantitative sequencing procedure may be utilized to identify the undigested molecular probes present in a sample by sequence, which may then be correlated to a target molecule for each of the particular molecular probes. So-called “next generation sequencing” systems, such as, for example, the Ion Torrent Personal Genome Machine (Life Technologies) or the Illumina Sequencer, may be utilized. The sequencing procedure may also utilize amplification steps, such as emulsion PCR, to, for example, increase signal from the molecular probes to, for example, increase the detection sensitivity of the assay.

In other embodiments, the assay may also be compatible with other readout systems and sequencing techniques. For example, methods of “in vitro compartmentalization” using reverse phase emulsions allow compartmentalized reactions accommodating a limited number of components. So-called “digital-PCR” may then be used with the assay in which an amplification of the nucleic acid molecular probe only occurs if it is protected with the emulsion compartment due to the presence of the corresponding target molecule. The compartmentalized reaction may generally indicate with high accuracy and precision the presence of the bound molecular probe and/or the number of bound molecular probes present.

In some embodiments, distinct droplets and/or emulsion droplets may be utilized with a plurality of molecular probes to different target molecules in each droplet. The droplets may also contain target molecules which may bind to the molecular probes. Each droplet may then be subjected to digestion of unbound molecular probes followed by amplification of the bound molecular probes in a digital PCR and/or other compartmentalized amplification reaction, such as above. Thus, each droplet may, after amplification, indicate the presence of a target in the droplet and/or the number of target molecules present initially in the droplet.

In another aspect of the invention, a normalizing or house-keeping target may be utilized to normalize the quantitation of detected molecular probes. In one embodiment, a house-keeping target may be present and/or introduced in a known amount and/or concentration in a sample such that a molecular probe that binds to the house-keeping target may be quantified along with the other molecular probes and thus used to normalize the quantitation.

The present invention together with the above and other advantages may best be understood from the following detailed description of the embodiments of the invention illustrated in the drawings.

### BRIEF DESCRIPTION OF THE FIGURES

FIG. 1 illustrates an overview of an assay with two targets in an embodiment of the invention.

### DETAILED DESCRIPTION OF THE INVENTION

The detailed description set forth below is intended as a description of the presently exemplified device provided in accordance with aspects of the present invention and is not intended to represent the only forms in which the present invention may be practiced or utilized. It is to be understood, however, that the same or equivalent functions and components may be accomplished by different embodiments that are also intended to be encompassed within the spirit and scope of the invention.

Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, the exemplified methods, devices and materials are now described.

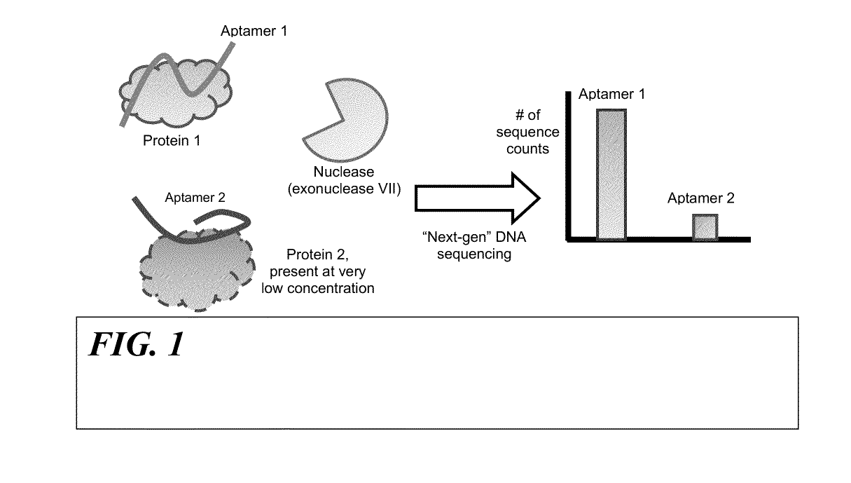

FIG. 1 illustrates an example of an assay in an embodiment of the present invention. In an aptamer-based, nuclease protection assay as illustrated, a cocktail of aptamers is digested by a single-strand-specific nuclease. Only those aptamers bound to their target protein survive digestion. The aptamers may further be modified to resist a particular form of nuclease degradation, such as 3′-degradation, and the nuclease utilized may be able to digest aptamers with the particular form of resistance, such as with a 5′-digesting nuclease. The remaining aptamer cocktail is then sequenced by standard methods on a “next generation DNA sequencer”. The number of counts of a particular aptamer sequence is directly proportional to the protein present in the sample. The assay is expected to be extremely sensitive and highly amenable to multiplexing (perhaps, for example, up to 100 protein targets).
1. Example to Demonstrate Protein Quantitation, Limit of Detection and Specificity in Single-Plex Assay

In Example 1 we will optimize the nuclease protection assay using an existing aptamer to penicillin binding protein 2a (PBP-2a) having an equilibrium dissociation constant (Kd) of 1.8 nM. We will establish the upper limit of the assay by the concentration of the aptamer before exposure to the protein sample—the [APTUL]. For various [APTUL] concentrations, various amounts of protein target will be spiked into the assay cocktail, and the lower limit of detection [LOD] will be determined. The “no-protein-control” (measured in triplicate) will establish the noise-floor of the assay. That is, incomplete digestion of the aptamer or spurious amplification may result in the persistence of some aptamer concentration from the original cocktail. Any [LOD] will be taken as at least 3 standard deviations above this noise floor. The coefficient of variation or CV at the [LOD] will also be measured from triplicate concentrations.

We will utilize standardized methods for sequencing the resulting barcoded library using an Ion Torrent Personal Genome Machine. Following sequencing, the entire dataset will be parsed first by the aptamer sequences determining the aptamer target in the assay cocktail. We will then count the frequency of expected sequences with 1, 2, and 3 mismatches and determine the effect of sequence quality on linearity of the sequence counts vs. protein concentration.
2. Example to Expand the Protocol to a Triplex Assay

At the completion of Example 1, we expect to have established a standard operating procedure for the reliable quantification of protein analytes in solution using our aptamer-based approach. There is however an undeniable advantage in being able to quantify multiple analytes over a single analyte in most prognostic and diagnostic assays. At the very least, most assays benefit from monitoring a standard housekeeping protein for internal validation. Using aptamers to cytokines IL-6 and IL-10, we will expand the single-plex assay developed in Example 1 into a triplex assay.

Independent standard curves. Informed from Example 1, we will first determine standard curves for the anti-IL6 and anti-IL10 aptamers independently (as for PBP-2a in Example I).

Titration of nuclease. In this experiment, we will determine the minimum amount of nuclease and digestion time required to completely eliminate (or at least minimize) the “noise floor” for an aptamer with no protein target present.

Effect of 2 unrelated aptamers on a single aptamer standard curve. In this experiment, we will examine the effect of 2 additional aptamers on the independent standard curve previously established.

TABLE 1
Experiments (rows) for assessment of aptamer
cross-reactivity to non-cognate targets.

| | | Protein | | |
|---|---|---|---|---|
| | | IL-6 | IL-10 | PBP2a |
| Aptamer | Expt #1: | NP | P | P |
| | a-IL-6 | | | |
| | Expt #2: | P | NP | P |
| | a-IL-10 | | | |
| | Expt #3: | P | P | NP |
| | a-PBP2a | | | |

P = protein present in a dilution series, NP = not present

Effect of multiplexing on assay independence. In the ideal case where all aptamer Kd's are equal, we can envision the scenario where all three upper limit, [APTUL]'s in the assay cocktail are 10 pM. In a simplified experiment, we will take just a single aptamer at that [APTUL] and expose it to the other two, non-cognate proteins (see Table 1). The non-cognate proteins will be offered in the same dilution series as in the independent standard curves and the effect of these proteins on the slope of the curve assessed. Any increase in slope or offset of the curve will quantitatively indicate binding of the aptamer to unintended target.

In a set of complementary experiments, we will assemble the full triplex cocktail of aptamers, and in a “leave-one-target-out” strategy, we will add 2 of 3 proteins and determine the influence of the added proteins on the independent calibration curves.

Quantitative Multiplexing and Normalization. Once we have established sufficient independence of the 3 systems above, we will determine the ability of our assay platform to detect all three protein targets at the same time with varying protein levels. While all three spiked-in protein concentrations will obviously be known, we will treat one protein as a spiked in external standard which we can normalize to. That is, in replicate measurements, will analyze the data both normalized and un-normalized in the anticipation that normalization to an externally spiked-in standard will remove any systematic errors in quantitation due to variables such as pipetting or differences in amplification efficiency. One may also envision an aptamer specific to a relatively constant “house-keeping” protein or albumin, for example, so that a second normalization might eventually be feasible in “real-world” samples.

3. Example to Test the Triplex Assay in Spiked Serum Samples

All of the above work in Examples 1 and 2 above will be performed in idealized conditions of PBS buffer. In order to build a genuinely-useful diagnostic assay based on our novel aptamer-exonuclease assay, we will demonstrate the detection of the 3 proteins above in a clinically relevant background (pooled human serum). The assay protocol will be identical to that outlined in Example 2. Pooled human serum will be obtained from Innovative Research (Novi, Mich.).

Because the MRSA protein, PBP-2a not expected to be present, this protein will be treated as a spiked-in control for normalization/quantitation. The IL-6 and IL-10 levels endogenous to the samples will be measured by our assay and compared to standard ELISAs for the proteins. In the event that the levels are undetectable by ELISA, the proteins will be spiked in to higher levels for comparison.

Additionally, the aptamers may be protected by modification of the 3′-end as discussed above.

4. Example of Cross-Linking Between Bound Aptamers and Target

In any of the forgoing examples, the aptamers bound to their targets may be crosslinked, as discussed above, to, for example, provide better protection against nuclease degradation of the bound aptamers while the unbound aptamers are digested. In aptamer to protein assays, we will use a reversible formaldehyde cross-linking reaction between aptamer and protein in order to achieve a more robust nuclease protection prior to amplification. Aptamers will be folded in 1 mM MgCl2 and 1×PBS pH=7.4 (Selection Buffer) by heating to 95° C. for 3 min and cooling to room temperature. Protein will then be added at a final concentration of 100 nM then allowed to bind at room temperature for 15 min. The aptamer:protein complex will then be cross-linked together by the addition of formaldehyde at a final concentration of 1%. After 10 min of incubation at room temperature, the reaction is quenched with 125 mM glycine. The aptamer:protein complex will then be digested with RQ1 DNase for min at 37° C. Finally, the sample is phenol chloroform extracted and the crosslinks reversed by heating at 70° C. for 4-5 hrs. The remaining undigested aptamers may then be amplified for the assay, as above.

5. Example of Sequencing of Assay Reaction

DNA sequencing may be accomplished using an Illumina MiSeq sequencing system. In this example, after second-strand synthesis of the aptamer pool samples, hairpin adapters will be ligated on to either end of the dsDNA (NEBNext Ultra kit). U-excision is performed to open the hairpin, followed by PCR amplification of the nascent library with barcoded oligos (NEBNext Multiplexed Adapters). The barcoded oligos enable multiplexing of samples within one sequencing run, bringing down costs by, for example multiplexing up to 24 samples in this fashion. Sequencing libraries will be quantified with qPCR (Kapa Biosystems), and then loaded onto the Illumina MiSeq system. After sequencing, the data will be demultiplexed and a bioinformatic analysis performed to determine the aptamer frequency relative to a control pool.

It will be appreciated by those of ordinary skill in the art that the present invention can be embodied in other specific forms without departing from the spirit or essential character hereof. The present description is therefore considered in all respects to be illustrative and not restrictive. The scope of the present invention is indicated by the appended claims, and all changes that come within the meaning and range of equivalents thereof are intended to be embraced therein.

1. A method for molecular detection comprising:
contacting a plurality of molecular probes with a sample for detecting the presence of at least one target in said sample, each of said molecular probes having binding affinity for a particular target;
digesting unbound molecular probes with a nuclease; and
performing a quantitative or semi-quantitative detection reaction on any undigested molecular probes;
wherein detection of any of said undigested molecular probes correlates to the presence of the associated targets in said sample.

2. The method of claim 1, further comprising an amplification reaction on any of said molecular probes bound to said targets.

3. The method of claim 2, wherein said amplification reaction comprises a compartmentalized amplification reaction.

4. The method of claim 1, wherein said molecular probes comprise nucleic acid aptamers.

5. The method of claim 4, wherein said nucleic acid aptamers comprise a modified 3′-prime nucleotide which is resistant to nuclease degradation.

6. The method of claim 5, wherein said modified 3′-prime nucleotide comprises 3′-prime inverted thymidine.

7. The method of claim 1, wherein said nuclease is selected to digest nucleic acids which are not bound to a target molecule.

8. The method of claim 7, wherein said nuclease comprises E. coli exonuclease VII.

9. A method for molecular detection comprising:
contacting a plurality of molecular probes comprising nucleic acids with a sample for detecting the presence of at least one target in said sample, each of said molecular probes having binding affinity for a particular target, said at least one target comprising a protein;
digesting unbound molecular probes with a nuclease; and
performing a quantitative or semi-quantitative detection reaction on any undigested molecular probes;
wherein detection of any of said undigested molecular probes correlates to the presence of the associated targets in said sample.

10. The method of claim 9, further comprising reversibly linking said molecular probe to said target.

11. The method of claim 10, wherein said reversibly linking comprises formaldehyde cross-linking.

12. The method of claim 10, wherein said linking is reversed after said digesting.

13. The method of claim 11, wherein said nuclease comprises RQ1 DNAse.

14. A method of molecular detection comprising:
contacting a plurality of molecular probes within one of a plurality of emulsion droplets with a sample, each of said molecular probes binding with specificity to a particular target;
digesting any of said molecular probes which do not bind to one of said particular targets with a nuclease; and
performing a compartmentalized amplification reaction on said plurality of emulsion droplets;
wherein said compartmentalized amplification reaction indicates the presence and number of molecules of said particular targets in each of said plurality of emulsion droplets.

15. The method of claim 14, wherein said compartmentalized amplification reaction comprises digital PCR.

16. The method of claim 14, wherein said nuclease is selected from the group consisting of E. coli exonuclease VII and RQ1 DNAse.

17. The method of claim 14, further comprising cross-linking any of said molecular probes to their said particular targets to which they are bound.

18. The method of claim 17, wherein said cross-linking comprises a reversible formaldehyde cross-linking reaction.

19. The method of claim 15, wherein said digital PCR comprises a correction for variations of the number of molecules of said particular targets present in each of said emulsion droplets.

20. The method of claim 17, wherein said cross-linking is reversed prior to said compartmentalized amplification reaction.
